SEQUENCE LISTING (6 sequences)

SEQ ID NO: 1 - RNA, 22 nt, Homo sapiens
uggaauguaaagaaguauguau

SEQ ID NO: 2 - RNA, 8 nt, Homo sapiens
acauucca

SEQ ID NO: 3 - DNA, 8 bp, Homo sapiens
acattcca

SEQ ID NO: 4 - RNA, 23 nt, Homo sapiens
gaaauacacaaguaaacauucca

SEQ ID NO: 5 - RNA, 22 nt, Homo sapiens
uauguaugaagaaauguaaggu

SEQ ID NO: 6 - RNA, 22 nt, Mus musculus
uauguaugaagaaauguaaggu

(19) United States
(12) Patent Application Publication
(10) Pub. No.: US 2023/0092762 A1
(43) Pub. Date: Mar. 23, 2023

(54) THERAPEUTIC DELIVERY OF LOCKED NUCLEIC ACID CONJUGATED ANTISENSE MIR-1

(71) Applicant: THE TRUSTEES OF INDIANA UNIVERSITY, Bloomington, IN (US)

(72) Inventors: Chandan K. SEN, Indianapolis, IN (US); Subhadip GHATAK, Indianapolis, IN (US)

(21) Appl. No.: 17/799,693

(22) PCT Filed: Feb. 23, 2021
(86) PCT No.: PCT/US2021/019190

§ 371 Date: Aug. 15, 2022

Related U.S. Application Data
(60) Provisional application No. 62/980,510, filed on Feb. 24, 2020.
(60) Provisional application No. 62/987,537, filed on Mar. 10, 2020.

(51) Int. Cl. A61K 31/7105; A61P 17/02; A61K 45/06

(52) U.S. Cl. CPC A61K 31/7105; A61P 17/02; A61K 45/06

(57) ABSTRACT

Compositions and methods are provided for promote wound healing in a subject by administering a miR-1 inhibitor to a wound on subject. In accordance with one embodiment such compositions are used in conjunction with known treatments for use on chronic wounds including in diabetic patients.

### CROSS REFERENCE TO RELATED APPLICATIONS

This application claims priority to the following: U.S. Provisional Patent Application No. 62/980,510 filed on Feb. 24, 2020 and U.S. Provisional Patent Application No. 62/987,537 filed on Mar. 10, 2020, the disclosure of which are expressly incorporated herein.

### INCORPORATION BY REFERENCES OF MATERIAL SUBMITTED ELECTRONICALLY

Incorporated by reference in its entirety is a computer-readable nucleotide/amino acid sequence listing submitted concurrently herewith and identified as follows: 5 kilobytes ACII (Text) file named “334052_ST25.txt,” created on Feb. 19, 2021.

### BACKGROUND OF THE DISCLOSURE

Nonhealing chronic wounds are a challenge to the patient, the health care professional, and the health care system. They significantly impair the quality of life for millions of people and impart a burden on society in terms of lost productivity and health care dollars. Peripheral vasculopathies, commonly associated with chronic wounds, are primarily responsible for wound ischemia. Limitations in the ability of the vasculature to deliver O₂-rich blood to the wound tissue leads to, among other consequences, hypoxia. Survival of the cutaneous wound tissue under ischemic conditions is dependent on biological responses aimed at minimizing the oxygen cost of survival by stabilizing hypoxia inducible factor (HIF).

Under conditions of ischemia, a number of microRNAs are induced by hypoxia driven transcription factors such as HIF-1α that brings down the cellular oxygen demand by curbing mitochondrial metabolism. Accordingly, hypoxia is a powerful stimulus regulating the expression of a specific subset of miRNAs, named hypoxia-induced miRNAs (hypoxamiR). These miRNAs are fundamental regulators of the cell responses to decreased oxygen tension. Although this mechanism is sustainable for a limited period, this is in direct conflict with the process of wound healing that relies on high supply of energy.

In the skin, notch ligand delta-like 1 (Dll1) is abundantly expressed in epidermis and is a recognized regulator of mitochondrial function. Loss of Dll1 may impede wound closure by delayed keratinocyte migration. Other aspects of wound healing such as angiogenesis and function of fibroblasts and platelets are also under direct control of the notch signaling pathway. Post-transcriptional gene silencing by miRNAs is of major significance in cutaneous wound healing. Anti-miR oligonucleotides have demonstrated benefits for wound closure in a number of experimental studies paving the way for more translational pursuit. A wide range of miR-directed oligonucleotides is currently in clinical development for the potential treatment of disorders. Encouragingly, FDA has approved oligonucleotide-based therapies and many more Investigational New Drug (IND) studies are in the pipeline making this line of pursuit translationally promising.

HypoxamiRs play a role in ischemic wound healing. HypoxamiRs involved in such processes are several and there is a delicate balance of multiple regulatory systems functioning in tandem to achieve tissue survival under conditions of ischemia. miR-1, otherwise recognized an myomiR, is a hypoxamiR that is induced in ischemic wounds of chronic wound patients as well as in an experimental animal model. Elevated miR-1 may blunt mitochondrial respiration, and therefore conserve oxygen consumption, under conditions of ischemia. While such mechanisms enable the survival of the ischemic wound tissue, sequestration of miR-1 is necessary to expedite rescue of wound healing.

### SUMMARY

In accordance with one embodiment of the present disclosure, a method of promoting wound healing in a subject is provided, the method comprising the step of administering a miR-1 inhibitor to a wound on subject. In accordance with one embodiment the method is directed to healing ischemic cutaneous wounds, including chronic wounds of diabetic patients. In one embodiment the method comprises administering an inhibitor that decreases functional miR-1 present in the cells of wound-edge tissue, thereby promoting wound healing. In one embodiment the the miR-1 inhibitor is an oligonucleotide, and more particularly is an antisense or interference RNA.

In one embodiment a method of enhancing wound closure in a subject and/or a stimulate keratinocyte proliferation and migration is provided. The method comprises the step of decreasing the abundance of functional miR-1 in the cells of wound-edge tissue by introducing an inhibitor of miR-1 into the cytosol of the cells of wound-edge tissues. In one embodiment the wound is an ischemic cutaneous wound, optionally wherein the wound to be treated is a chronic wound in a diabetic patient. In one embodiment the inhibitor of miR-1 is administered to wound-edge tissue in an amount effective to lower miR-1 activity and increase Dll1 activity.

In one embodiment the miR-1 inhibitor is an oligonucleotide at least 8 nucleotides in length, wherein the oligonucleotide has at least 80%, 85%, 95% or 99% sequence identity to a continuous 8 nucleotide sequence of human mature miR-1 sequence (SEQ ID NO: 1) or a complement thereof. In one embodiment the miR-1 inhibitor is an oligonucleotide at least 8 nucleotides in length, wherein 8 nucleotides of the oligonucleotide has 100% sequence identity to a continuous 8 nucleotide sequence of human mature miR-1 sequence (SEQ ID NO: 1) or a complement thereof. In one embodiment the oligonucleotide comprises the sequence of ACAUUCCA (SEQ ID NO: 2), or its complement. In one embodiment the miR-1 inhibitor is an RNA comprising a locked nucleic acid, optionally wherein the locked nucleic acid is the N-terminal or C-terminal nucleotide of the oligonucleotide, or is present at both the N-terminus and the C-terminus of the oligonucleotide.

In accordance with one embodiment the miR-1 inhibiting oligonucleotide is introduced into the cytosol of cells using any transfection technique known to those skilled in the art. Known delivery methods can be broadly classified into two types. In the first type, a membrane-disruption-based method involving mechanical, thermal or electrical means can be used to disrupt the continuity of the cell membrane with enhanced permeabilization for direct penetration of desired macromolecules. In the second type, a carrier-based method, using various viruses, exosomes, vesicles and nanoparticle capsules, allows uptake of the carrier through endocytosis and fusion processes of cells for delivery of the carrier payload. In accordance with one embodiment the miR-1 inhibiting oligonucleotide is administered in an amount to effectively reduce functional miR-1 concentrations and increase Dll1 activity by transfecting cells with the oligonucleotide. In one embodiment the anti-miR-1 oligonucleotide is delivered into the cytosol of human keratinocytes. In one embodiment the oligonucleotide is delivered into the cytosol of cells via skin electroporation or tissue nanotransfection.

In one embodiment a pharmaceutical composition for enhancing wound closure is provided, wherein said composition comprises an oligonucleotide at least 8 nucleotides in length, wherein the oligonucleotide has at least 85% sequence identity to a continuous 8 nucleotide sequence of human mature miR-1 sequence (SEQ ID NO: 1) or a complement thereof and a pharmaceutically acceptable carrier.

### BRIEF DESCRIPTION OF THE DRAWINGS

FIGS. 1A-1D illustrate that ischemia induces miR-1 expression. FIG. 1A is a bar graph presenting the expression of miR-1 in human healing and non-healing (open >30 days) wound-edge tissue. (n=3). FIG. 1B is a bar graph presenting the results of RT-qPCR of miR-1 expression in ischemic and non-ischemic wound-edge tissue from C57BL/6 mice at day 3 post-wounding. (n=7). FIG. 1C is a bar graph presenting the expression of miR-1 in human keratinocytes (HaCaT cells) subjected to normoxia (20% O₂) and hypoxia (5% O₂) for 24 h (n=6). FIG. 1D is a bar graph presenting the expression of miR-1 in 72 h post-infection with AdVP16 (control) or AdVP16-HIF-1α viral vectors for forced stabilization of HIF-1α under normoxic condition (n=4). Data shown as mean±SD; *, p<0.05; **, p<0.01; ***, p<0.001; ANOVA.

FIGS. 2A-2D illustrate that miR-1 targets Dll-1. FIG. 2A is a schematic drawing demonstrating the possible binding site in Dll1 3′-UTR (SEQ ID NO: 4) for human (has-miR-1; SEQ ID NO: 5) and mouse (mmu-miR-1; SEQ ID NO: 6) miR-1 as predicted by Targetscan. FIG. 2B is a bar graph presenting the result of an miRNA target reporter luciferase assay after miR-1 mimic delivery in HaCaT cells. Open and solid bars represent control mimic and miR-1 mimic-delivered cells, respectively. Results were normalized with Renilla luciferase (n=5). FIGS. 2C and 2D present Western blot analysis and quantitation of Dll1 expression in human keratinocytes after transfection with miR-1 mimic (n=3; FIG. 2C) and miR-1 inhibitor (n=6; FIG. 2D). Data shown as mean±SD; *, p<0.05; ***, p<0.001; ANOVA.

FIGS. 3A-3C illustrate that downregulation of miR-1 and upregulation of Dll1 is critical for wound closure. FIG. 3A. is a schematic representation of a mouse model of excisional wounding procedure and a graph demonstrating that miR-1 (n=4) concentrations decreased over time after injury, as detected using RT-qPCR of miR-1. FIG. 3B presents Western blot analysis and quantitation of Dll1 expression in skin, and d3, d7 and d14 non-ischemic wound-edge tissue (n=3). FIG. 3C presents Western blot and quantitation of Dll1 expression in skin, non-ischemic and ischemic wound-edge tissue at day 7 post-wounding (n=3). Data shown as mean±SD; *, p<0.05; **, p<0.01; ***, p<0.001; ANOVA.

FIGS. 4A & 4B illustrate that miR-1 impaired cell migration and cell proliferation. Human keratinocytes were transfected with control and miR-1 mimic for 48 h followed by scratch assay. FIG. 4A is a bar graph presenting the measurement of keratinocyte migration, expressed as percentage closure at 5 h and 10 h following scratch (n=5). FIG. 4B is a bar graph presenting the results of a MTT assay of keratinocytes transfected with control and miR-1 mimic after 48 h (n=6). Data shown as mean±SD; **, p<0.01; ***, p<0.001; ANOVA.

FIGS. 5A-5D illustrate that miR-1 triggered downregulation of Dll1, induces mitochondrial depolarization and subsequent cell death. FIG. 5A is a graph presenting data on oxygen consumption of control and miR-1 mimic transfected human keratinocytes. Cells were seeded in a 96 well plate for real-time assessment of OCR in a XF-96 Sea Horse analyzer (n=10). FIG. 5B presents the ATP:ADP ratio in the cells, quantified from human keratinocytes 48 h after transfection of control and miR-1 mimics and plotted graphically (n=6). Evaluation of mitochondrial membrane potential (ΔΨ) changes in human keratinocyte transfected with either control or miR-1 mimic was assessed by JC-1 flow cytometry 48 h post-transfection. Significantly increase in JC-1 green fluorescence in keratinocytes was observed with compromised ΔΨ. The percentage of JC-1 green fluorescence was plotted graphically (n=5; see FIG. 5C). Human keratinocytes transfected with either control or miR-1 mimic. The transfected cells were stained with tetramethylrhodamine methyl ester (TMRM) and plasma membrane potential indicator (PMPI) simultaneously. The intensity of TMRM fluorescence was plotted graphically (n=6; see FIG. 5D). Data shown as mean±SD; *, p<0.05; **, p<0.01; ***, p<0.001; ANOVA.

FIGS. 6A-6D show that pharmacological inhibition of Dll1 impairs mitochondrial depolarization. Assessment of OCR after treatment with Notch signaling inhibitor MK-0752 (20 μM, 24 h) depicts the direct role of notch signaling (n=10; see FIG. 6A). Evaluation of mitochondrial membrane potential (ΔΨ) changes treated with MK-0752 (20 μM, 24 h), showed significant increase in JC-1 green fluorescence in keratinocytes with compromised ΔΨ, with FIG. 6B providing a graph of the percentage of JC-1 green fluorescence (n=4). FIG. 6C provide a graph of the TMRM and PMPI staining results, demonstrating significant decrease in TMRM fluorescence in human keratinocytes after MK-0752 treatment (n=9). Western blot analysis and quantitation of BAX expression in human keratinocytes 24 h after treatment with 20 μM of MK-0752 is provided in FIG. 6D (n=3). Data shown as mean±SD; *, p<0.05; ***, p<0.001; ANOVA.

FIGS. 7A-7E show that delivery of anti-miR-1 facilitates ischemic wound closure in mice. Digital photographs of ischemic wound were taken seven days after wounding in scramble oligos (control) and LNA anti-miR-1 delivered group. FIG. 7A is a graphic representation of the wound closure in the control and test samples (n=4). Serial wound cross-sections were stained with anti-Keratin 14 antibody and counter stained with DAPI (blue) (n=4). Scale bar=1000 μm. The percentage of re-epithelialization was plotted (n=3) as shown in FIG. 7B. Serial wound cross-sections were stained with anti-Ki67 antibody and counter stained with Hematoxylin (blue), Scale bar=50 μm. Quantification of Ki67 positive cells (brown) is provided graphically in FIG. 7C (n=6). Laser speckle image was used to measure perfusion level in the bipedicle flap at day 0 and day 7 after delivery of scramble oligos (control) and LNA anti miR-1. Quantification of perfusion is shown graphically (n=3) in FIG. 7D. Western blot analysis of BAX from day 7 wound-edge tissue samples is provided in FIG. 7E (n=3). Data shown as mean±SD; *, p<0.05; **, p<0.01; ***, p<0.001; ANOVA.

FIGS. 8A-8B show results of human keratinocyte transfection and OCR assessment. FIG. 8A shows the results for human keratinocytes transfected with either control and miR-1 mimic seeded in a 96 well plate and analyzed using real-time assessment of OCR in a XF-96 Sea Horse analyzer. Significant reduction in basal respiration and ATP production was observed compare to control mimic. Assessment of OCR after treatment with vehicle control and MK-0752 (20 μM, 24 h) showed significant decrease in basal respiration and ATP production (FIG. 8B). Data expressed as mean t SD (n=10, ** p<0.01 *** p<0.001).

### DETAILED DESCRIPTION

### Definitions

In describing and claiming the invention, the following terminology will be used in accordance with the definitions set forth below.

The term “about” as used herein means greater or lesser than the value or range of values stated by 10 percent but is not intended to limit any value or range of values to only this broader definition. Each value or range of values preceded by the term “about” is also intended to encompass the embodiment of the stated absolute value or range of values.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition. The term “purified polypeptide” is used herein to describe a polypeptide which has been separated from other compounds including, but not limited to nucleic acid molecules, lipids and carbohydrates.

The term “isolated” requires that the referenced material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

Tissue nanotransfection (TNT) is an electroporation-based technique capable of delivering nucleic acid sequences and proteins into the cytosol of cells at nanoscale. More particularly, TNT uses a highly intense and focused electric field through arrayed nanochannels, which benignly nanoporates the juxtaposing tissue cell members, and electrophoretically drives cargo (e.g., nucleic acids or proteins) into the cells.

As used herein a “control element” or “regulatory sequence” are non-translated regions of a functional gene, including enhancers, promoters, 5′ and 3′ untranslated regions, which interact with host cellular proteins to carry out transcription and translation. Such elements may vary in their strength and specificity. “Eukaryotic regulatory sequences” are non-translated regions of a functional gene, including enhancers, promoters, 5′ and 3′ untranslated regions, which interact with host cellular proteins of a eukaryotic cell to carry out transcription and translation in a eukaryotic cell including mammalian cells.

As used herein a “promoter” is a sequence or sequences of DNA that function when in a relatively fixed location in regard to the transcription start site of a gene. A “promoter” contains core elements required for basic interaction of RNA polymerase and transcription factors and can contain upstream elements and response elements.

As used herein an “enhancer” is a sequence of DNA that functions independent of distance from the transcription start site and can be either 5′ or 3′ to the transcription unit. Furthermore, enhancers can be within an intron as well as within the coding sequence itself. They are usually between 10 and 300 bp in length, and they function in cis. Enhancers function to increase transcription from nearby promoters. Enhancers, like promoters, also often contain response elements that mediate the regulation of transcription. Enhancers often determine the regulation of expression.

An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter. As used herein an exogenous sequence in reference to a cell is a sequence that has been introduced into the cell from a source external to the cell.

As used herein the term “non-coded (non-canonical) amino acid” encompasses any amino acid that is not an L-isomer of any of the following 20 amino acids: Ala, Cys, Asp, Glu, Phe, Gly, His, Ile, Lys, Leu, Met, Asn, Pro, Gln, Arg, Ser, Thr, Val, Trp, Tyr.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity. Those skilled in the art will recognize that several computer programs, such as those that employ algorithms such as BLAST (Basic Local Alignment Search Tool, Altschul et al. (1993) J. Mol. Biol. 215:403-410) are available for determining sequence identity.

The term “stringent hybridization conditions” as used herein mean that hybridization will generally occur if there is at least 95% and preferably at least 97% sequence identity between the probe and the target sequence. Examples of stringent hybridization conditions are overnight incubation in a solution comprising 50% formamide, 5×SSC (150 mM NaCI, 15 mM trisodium citrate), 50 mM sodium phosphate (pH 7.6), 5×Denhardt's solution, 10% dextran sulfate, and 20 μg/ml denatured, sheared carrier DNA such as salmon sperm DNA, followed by washing the hybridization support in 0.1×SSC at approximately 65° C. Other hybridization and wash conditions are well known and are exemplified in Sambrook et al, Molecular Cloning: A Laboratory Manual, Second Edition, Cold Spring Harbor, N.Y. (1989), particularly chapter 11.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “phosphate buffered saline” or “PBS” refers to aqueous solution comprising sodium chloride and sodium phosphate. Different formulations of PBS are known to those skilled in the art but for purposes of this invention the phrase “standard PBS” refers to a solution having have a final concentration of 137 mM NaCl, 10 mM Phosphate, 2.7 mM KCl, and a pH of 7.2-7.4.

As used herein, the term “treating” includes prophylaxis of the specific disorder or condition, or alleviation of the symptoms associated with a specific disorder or condition and/or preventing or eliminating said symptoms.

As used herein an “effective” amount or a “therapeutically effective amount” of a drug refers to a nontoxic but enough of the drug to provide the desired effect. The amount that is “effective” will vary from subject to subject or even within a subject overtime, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein an amino acid “substitution” refers to the replacement of one amino acid residue by a different amino acid residue.

As used herein, the term “conservative amino acid substitution” is defined herein as exchanges within one of the following five groups:
I. Small aliphatic, nonpolar or slightly polar residues:
   Ala, Ser, Thr, Pro, Gly;
II. Polar, negatively charged residues and their amides:
   Asp, Asn, Glu, Gln;
III. Polar, positively charged residues:
   His, Arg, Lys; Ornithine (Orn)
IV. Large, aliphatic, nonpolar residues:
   Met, Leu, Ile, Val, Cys, Norleucine (Nle), homocysteine (hCys)
V. Large, aromatic residues:
   Phe, Tyr, Trp, acetyl phenylalanine, napthylalanine (Nal)

As used herein the term “patient” without further designation is intended to encompass any warm blooded vertebrate domesticated animal (including for example, but not limited to livestock, horses, cats, dogs and other pets) and humans and includes individuals not under the direct care of a physician.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini.

The term “amino acid sequence” refers to a series of two or more amino acids linked together via peptide bonds wherein the order of the amino acids linkages is designated by a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

“Nucleotide” as used herein is a molecule that contains a base moiety, a sugar moiety, and a phosphate moiety. Nucleotides can be linked together through their phosphate moieties and sugar moieties creating an internucleoside linkage. The term “oligonucleotide” is sometimes used to refer to a molecule that contains two or more nucleotides linked together. The base moiety of a nucleotide can be adenine-9-yl (A), cytosine-1-yl (C), guanine-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide that contains some type of modification to the base, sugar, and/or phosphate moieties. Modifications to nucleotides are well known in the art and would include, for example, 5-methylcytosine (5-me-C), 5 hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but are linked together through a moiety other than a phosphate moiety. Nucleotide substitutes are able to conform to a double helix type structure when interacting with the appropriate target nucleic acid.

The term “vector” or “construct” designates a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “operably linked to” refers to the functional relationship of a nucleic acid with another nucleic acid sequence. Promoters, enhancers, transcriptional and translational stop sites, and other signal sequences are examples of nucleic acid sequences that can operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

As used herein “Interfering RNA” is any RNA involved in post-transcriptional gene silencing, which definition includes, but is not limited to, double stranded RNA (dsRNA), small interfering RNA (siRNA), and microRNA (miRNA) that are comprised of sense and antisense strands.

As used herein a “locked nucleic acid” (LNA), is a modified RNA nucleotide in which the ribose moiety is modified with an extra bridge connecting the 2′ oxygen and 4′ carbon. For example, a locked nucleic acid sequence comprises a nucleotide of the structure:

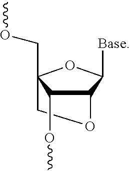

As used herein the term “vasculogenesis” is defined as the differentiation of precursor cells (angioblasts) into endothelial cells and the de novo formation of a primitive vascular network.

As defined herein “wound healing” defines a process wherein a living organism replaces destroyed or damaged tissue by newly produced tissue. The process includes three phases blood clotting, tissue growth (cell proliferation), and tissue remodeling. Accelerated wound healing includes a shorten length of time required to complete any of three phases, including for example the closure of an open wound due to tissue growth.

### EMBODIMENTS

The adaptive responses to hypoxia are multifaceted and primarily aimed at survival of the affected tissue. Inducible expression of hypoxamiRs also serves that purpose by turning down mitochondrial respiration thus conserving tissue oxygen to support other vital processes necessary for survival. While such dampening of oxidative metabolism is productive under acute conditions, sequestration of hypoxamiRs may be necessary to resume key physiological processes such as wound healing. Deleterious effects of hypoxamiRs have been evident in a wide range of other pathophysiological conditions. Strategies to inhibit hypoxamiR function are therefore of interest in the context of such conditions.

Otherwise widely recognized as a myomiR, miR-1 is a HIF1α-dependent hypoxamiR. Abundance of miR-1 is known to be detrimental to adult cardiac myocytes. Under normal conditions, elevated miR-1 results in arrhythmia due to compromised intracellular calcium trafficking system. In silico studies predicted that the 3′-UTR of notch ligand Dll1 is a likely target of miR-1. In the skin, Dll1 is abundantly expressed in keratinocytes. Dll1 signaling is critical for providing a steady supply of energy and maintaining adequate cellular metabolism. The translocation of pro-apoptotic BAX from the cytoplasm to the outer membranes of the mitochondria compromises membrane integrity. Loss of mitochondrial integrity causes a drop in the mitochondrial potential and subsequently the cellular energy state thereby stalling all the active energy-dependent processes such as proliferation and migration. Dll1 signaling prevents BAX dimerization on the mitochondrial membrane.

Thus, under conditions of hypoxia, tissue survival depends on a fine balance between hypoxia-inducible miR-1 and retention of its target Dll1. Deficiencies in Dll1 signaling leads to tissue necrosis with loss of limb in a hindlimb model of ischemia. Long-term abundance of miR-1 is in direct conflict with the anabolism of tissue that is known to be largely dependent on mitochondrial function and oxidative metabolism. Observation of this work that in the ischemic wound miR-1 sequestration improves healing outcomes lends credence to this notion. miR-1 sequestration is also likely to enable insulin growth factor-1 (IGF-1) signaling, a well-known mechanism implicated in cutaneous wound healing. In addition to its beneficial effect on wound healing, IGF-1 signaling defends against oxidative stress-induced mitochondrial dysfunction, cytochrome-c release and apoptosis.

Notch receptor is a type I membrane precursor heterodimer that is subject to two subsequent cleavages induced by the engagement of its ligand. Such receptor-ligand binding releases a functional intracellular form of Notch. Prevention of ligand-inducible receptor cleavage at the cell surface with γ-secretase inhibitors represents a robust approach to inhibit Notch-Dll1 signaling. The γ-secretase inhibitor MK-0752 was tested to investigate the significance of Dll1 signaling in ischemic wound healing. Significance of this line of study is further enhanced by the observation that MK-0752, also known as Taxotere or Neulasta, has been successfully tested in a phase I trial showing reduction of breast cancer stem cells. Breast cancer patients often suffer from ischemic radiation ulcers. Other examples of drugs that are widely used for treating cancer but may be detrimental for wound healing include cyclophosphamide, thiotepa, mechlorethapine and cisplatin. These drugs prevent neovascularization and inhibit fibroblast function. Other cancer drugs such as methotrexate, 5-fluorouracil, bleomycin and actinomycin D are known to limit collagen production and thus compromise wound tensile strength.

In accordance with the present disclosure miR-1 is recognized as an HF1 α-dependent hypoxamiR in the ischemic wound edge tissue. In keratinocytes, miR-1 silences notch ligand Dll1 by binding to its 3′UTR. Low Dll1 compromises mitochondrial function. Thus, key physiological processes critical for re-epithelialization, such as keratinocyte proliferation and migration are severely compromised. Pharmacological inhibition of Notch-Dll1 signaling using MK-0752 has also been disclosed herein to impair wound healing. Because this drug is under development for the treatment of breast cancer, specific attention to complicated wound healing outcomes in such patients is warranted and complementary treatment with therapeutics that sequester or inhibit excessive miR-1 may be appropriate. In one embodiment strategies to sequester or inhibit excessive miR-1 in the wound-edge tissue are used for the therapeutic management of ischemic wounds.

In accordance with one embodiment, a method is provided for promoting wound healing in a subject by administering a therapeutic agent that reduces miR-1 activity. In one embodiment a miR-1 inhibitor is brought in contact with a wound on subject, in an amount effective to reduce the function or activity of miR-1, thereby promoting wound healing. In one embodiment miR-1 inhibitor is delivered locally to the wound by physical contact of a topical formulation, or by injection of an miR-1 inhibitor into wound-edge tissue. In one embodiment, the miR-1 inhibitor is administered by skin electroporation or tissue nanotransfection. In one embodiment, the miR-1 inhibitor is an oligonucleotide, including for example an oligonucleotide comprising a locked nucleic acid (LNA) conjugated antisense miR-1 oligonucleotide.

The miR-1 inhibitor oligonucleotides disclosed herein may comprise one or more locked nucleic acid (LNAs) residues, or “locked nucleotides.” The oligonucleotides of the present invention may comprise one or more nucleotides containing other sugar or base modifications. The terms “locked nucleotide,” “locked nucleic acid unit,” “locked nucleic acid residue,” “LNA” or “LNA unit” may be used interchangeably throughout the disclosure and refer to a bicyclic nucleoside analogue. For instance, suitable oligonucleotide inhibitors can be comprised of one or more “conformationally constrained” or bicyclic sugar nucleoside modifications (BSN) that confer enhanced stability to complexes formed between the oligonucleotide containing BSN and their complementary target strand.

In one embodiment the miR-1 inhibitory oligonucleotide may comprise, consist essentially of, or consist of, an interference RNA or antisense sequence to miR-1. In one embodiment, the oligonucleotide comprises an antisense sequence directed to miR-1. For example, the oligonucleotide can comprise a sequence of at least 8 nucleotides that has at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a continuous 8 nucleotide sequence of human mature miR-1 sequence (SEQ ID NO: 1). In one embodiment, the oligonucleotide inhibitor as provided herein comprises a sequence that has 100% sequence identity (i.e., fully complementary) with a contiguous sequence found within the mature miR-1 sequence. It is understood that the sequence of the oligonucleotide inhibitor is considered to be complementary to miR-1 even if the oligonucleotide inhibitor sequence includes a modified nucleotide instead of a naturally-occurring nucleotide.

In one embodiment the oligonucleotide miR-1 inhibitor is an RNA 8-15 nucleotide in length and comprising a sequence that has at least 80, 85, 90, 95 or 99% sequence identity with a contiguous sequence found in the miR-1 sequence of SEQ ID NO: 1. In one embodiment the oligonucleotide miR-1 inhibitor is an RNA comprising the sequence ACAUUCCA (SEQ ID NO: 2), or its complement, or the corresponding DNA (ACATTCCA (SEQ ID NO: 3), or its complement). In one embodiment any of the oligonucleotide miR-1 inhibitors disclosed herein further comprises a locked nucleic acid. In one embodiment the oligonucleotide comprises two or more locked nucleic acids. In one embodiment the oligonucleotide miR-1 inhibitor is an RNA comprising
i) a single locked nucleic acid at its 5′ terminus;
ii) a single locked nucleic acid at its 3′ terminus; or
iii) a locked nucleic acid at its 5′ and 3′ terminus.
   In one embodiment the oligonucleotide miR-1 inhibitor is an RNA comprising the sequence ACAUUCCA (SEQ ID NO: 2), or its complement, and an additional locked nucleic acid, located at its 5′ terminus or 3′ terminus or at both the 5′ terminus and the 3′ terminus.

The wound to be treated in accordance with the present disclosure may be a surgical wound, a chronic wound, or an acute wound. In addition, the wound may be an incision, a pressure ulcer, a venous ulcer, an arterial ulcer, a diabetic lower extremity ulcer, a laceration, an abrasion, a puncture, a contusion, an avulsion, a cavity, a burns, or any combination thereof. The wound may be a wound edge, a wound bed, and/or a peri-wound.

In one embodiment, a method of promoting wound healing in a subject comprises administering to the subject a miR-1 inhibitor, such as an oligonucleotide disclosed herein. In some embodiments, the subject suffers from diabetes. In some embodiments, healing of a chronic wound, diabetic foot ulcer, venous stasis leg ulcer or pressure sore is promoted by administration of a miR-1 inhibitor.

In one embodiment, administration of a miR-1 inhibitor as provided herein provides at least about 5%, 10%, 20%, 30%, 40%, 50%, 60%, 70%, 80%, or 90% improvement in wound re-epithelialization or wound closure as compared to a wound not administered the miR-1 inhibitor relative to time. In some embodiments, administration of a miR-1 inhibitor as provided herein provides at least about 5%, 10%, 20%, 30%, 40%, 50%, 60%, 70%, 80%, or 90% more granulation tissue formation or neovascularization as compared to a wound not administered the miR-1 inhibitor.

In one embodiment, administration of a miR-1 inhibitor as provided herein provides at least about 5%, 10%, 20%, 30%, 40%, 50%, 60%, 70%, 80%, or 90% improvement in wound re-epithelialization or wound closure as compared to a wound administered an agent known in the art for treating wounds relative to time. In some embodiments, administration of a miR-1 inhibitor as provided herein provides at least about 5%, 10%, 20%, 30%, 40%, 50%, 60%, 70%, 80%, or 90% more granulation tissue formation or neovascularization as compared to a wound administered an agent known in the art for treating wounds relative to time.

In accordance with the present invention nucleic acids and/or proteins are introduced into the cytosol of cells of wound-edge tissue, including for example dermal fibroblasts, to decrease the concentration of function miR-1 in the target cells. Any of the standard techniques for introducing macromolecules into cells can be used in accordance with the present disclosure. Known delivery methods can be broadly classified into two types. In the first type, a membrane-disruption-based method involving mechanical, thermal or electrical means can be used to disrupt the continuity of the cell membrane with enhanced permeabilization for direct penetration of desired macromolecules. In the second type, a carrier-based method, using various viruses, exosomes, vesicles and nanoparticle capsules, allows uptake of the carrier through endocytosis and fusion processes of cells for delivery of the carrier payload.

In one embodiment intracellular delivery is via a viral vector, or other delivery vehicle capable of interacting with a cell membrane to deliver its contents into a cell. In one embodiment intracellular delivery is via three-dimensional nanochannel electroporation, delivery by a tissue nanotransfection device, or delivery by a deep-topical tissue nanoelectroinjection device. In one embodiment the miR-1 inhibitor is delivered into the cytosol of cells of wound-edge tissues in vivo through tissue nanotransfection (TNT) using a silicon hollow needle array.

Among the methods of permeabilization-based disruption delivery, electroporation has already been established as a universal tool. High efficiency delivery can be achieved with minimum cell toxicity by careful control of the electric field distribution. In accordance with one embodiment nucleic acid sequences are delivered to the cytosol of somatic cells through the use of tissue nanotransfection (TNT). Tissue nanotransfection (TNT) is an electromotive gene transfer technology that delivers plasmids, RNA and oligonucleotides to live tissue causing direct conversion of tissue function in vivo under immune surveillance without the need for any laboratory procedures. Unlike viral gene transfer commonly used for in vivo tissue reprogramming, TNT obviates the need for a viral vector and thus minimizes the risk of genomic integration or cell transformation.

Current methods can involve transfecting cells in vivo or in vitro followed by implantation. Although one embodiment of the present invention entails in vitro transfection of cells followed by transplantation, cell implants are often met with low survival and poor tissue integration. Additionally, transfecting cells in vitro involves additional regulatory and laboratory hurdles.

In accordance with one embodiment the cells of wound-edge tissue are transfected in vivo with an miR-1 interference oligonucleotide comprising composition as disclosed herein. Common methods for bulk in vivo transfection are delivery of viral vectors or electroporation. Although viral vectors can be used in accordance with the present disclosure for delivery of a oligonucleotides, viral vectors suffer the drawback of potentially initiating undesired immune reactions. In addition, many viral vectors cause long term expression of gene, which is useful for some applications of gene therapy, but for applications where sustained gene expression is unnecessary or even undesired, transient transfection is a viable option. Viral vectors also involve insertional mutagenesis and genomic integration that can have undesired side effects. However, in accordance with one embodiment certain non-viral carriers, such as liposomes or exosomes can be used to deliver a miR-1 interference oligonucleotide to somatic cells in vivo.

TNT provides a method for localized gene delivery that causes direct transfection of tissues in vivo under immune surveillance without the need for any laboratory procedures. By using TNT with oligonucleotides or plasmids, it is possible to temporally and spatially control overexpression of a gene or inhibit expression of a target gene. Spatial control with TNT allows for transfection of a target area such as a portion of skin tissue without transfection of other tissues. Details regarding TNT devices have been described in US published patent application nos. 20190329014 and 20200115425, the disclosures of which are expressly incorporated by reference.

Tissue nanotransfection allows for direct cytosolic delivery of cargo (e.g., interference oligonucleotides or genes) into cells by applying a highly intense and focused electric field through arrayed nanochannels, which benignly nanoporates the juxtaposing tissue cell members, and electrophoretically drives cargo into the cells.

### Example 1

LNA Conjugated Anti-mIR-1 Enhances Ischemic Wound Closure

Cell and cell culture and hypoxic treatment. Immortalized human keratinocytes (HaCaT) cells were maintained under the conditions known to those skilled in the art. In brief, HaCaT cells were maintained in DMEM (Life Technologies, Grand Island, N.Y.) supplemented with 10% fetal bovine serum, 100 U/ml penicillin, and 0.1 mg/ml streptomycin (Invitrogen Life Technologies), and incubated at 37° C. and 5% CO₂ in humidified chamber. For hypoxic treatment, cells were seeded on a 35-mm dish at 0.5×10⁶ cells/plate 1 day before hypoxic treatment. Medium was refreshed and incubated at either normoxic (20% O₂) or hypoxic (1% O₂) in a chamber with the same humidity and temperature as described. After 24 h, cells were lysed and RNA was extracted as described later.

Transfection of miRNA mimic and inhibitors. HaCaT cells (0.05×10⁶ cells/well in 12-well plate) were seeded in antibiotic free medium for 18-24 h prior to transfection. DharmaFECT™ 1 transfection reagent was used to transfect cells with miRIDIAN hsa-miR-1 mimic (50 nM), hsa-miR-1 inhibitor (100 nM, Thermo Scientific Dharmacon RNA Technologies, Lafayette, Colo.) per the manufacturer's instructions. miRIDIAN miR mimic/inhibitor negative controls (Thermo Scientific Dharmacon RNA Technologies, Lafayette, Colo.) were used for control transfections. Samples were collected after 48 h of miR mimic or 72 h of miR inhibitor transfection for quantification of miRNA or protein expression.

miRNA target reporter luciferase assay. HaCaT cells, transfected with control and miR mimic for 48 hours, were transfected with 500 ng/sample of pLuc-Notch1 3′-UTR plasmid (GeneCopoeia, Inc, Rockville, Md.) or control construct together with Renilla luciferase pRL-cmv expression construct (10 ng/sample) using Lipofectamine™ LTX PLUS™ reagent (Life Technologies, Grand Island, N.Y.) according to the manufacturer's protocol. Luciferase activity was determined 24 hours after transfection. After 24 h, cell lysates were assayed with dual luciferase reporter assay kit (Promega, Madison, Wis.). The data are presented as ratio of firefly to Renilla luciferase activity.

RNA extraction and quantitative real-time PCR. RNA from mouse wound-edge tissue (d3) or cells were isolated using miRVana miRNA Isolation Kit according to the manufactures protocol (Ambion Life Technologies). The RNA quality was assessed using Agilent 2100 Bioanalyzer (Agilent Technologies, Santa Clara, Calif.). For determination of miR expression, specific TaqMan assays for miRs and the TaqMan miRNA reverse transcription kit were used, followed by real-time PCR using the Universal PCR Master Mix (Applied Biosystems, Foster City, Calif.) mRNA was quantified by real-time or quantitative (Q) PCR assay using the double-stranded DNA binding dye SYBR Green-I as described previously. The data were normalized against U6 miRNA.

Respirometry Assay. The Oxygen Consumption Rate (OCR) measurements were performed using a Seahorse Bioscience XF-96 instrument. A day prior to the experiment, the sensor cartridge was hydrated overnight using the calibration buffer supplied by the manufacturer. The transfected cells were seeded in the 96 well microplate supplied by the company. On the day of the experiment, the cells were washed with the calibrant buffer twice and incubated with glucose free DMEM for 2 h at 37° C. in a CO₂ free incubator. The injection ports of the sensors were filled with 20 μL of treatment or vehicle in buffer. The sensor was then placed into the XF-96 instrument and calibrated. After calibration, the calibration fluid plate was replaced with the cell plate. The measurement cycle consisted of a 2 min mix, 1 min wait, and a 2 min measurement. Four basal rate measurements were followed with injections and each injection is followed by four measurement cycles. The consumption rates were calculated from the continuous average slope of the O₂ decreases using a compartmentalization model. For any one treatment, the rates from 10 wells were used. Rates for the wells were normalized for protein content. All average rates were normalized to the vehicle control basal and t-tests between control and treatment were used to assess statistical significance.

Scratch assay. A cell migration assay was performed using culture inserts (IBIDI, Verona, Wis.) according to the manufacturer's instructions. Briefly, confluent cellular monolayer is formed in the presence of the insert inside a chamber. Removal of the insert generated a gap in the monolayer. Migration of cells across that gap was studied using time-lapse microscopy. As required, cells were transfected with either control or miR-1 mimic. Cell migration was measured using timelapse phase-contrast microscopy following withdrawal of the insert. Images were analyzed using the AxioVision Rel 4.8 software. Extent of migration in control and miR-1 mimic transfected cells were analyzed after 5 and 10 h after transfection.

Measurement of Mitochondrial Membrane Potential. Mitochondrial membrane potential changes were assessed by two different techniques, a) using the lipophilic cationic dye JC-1 (MitoProbe JC-1 Assay Kit for Flow Cytometry, Life technologies) per manufacturer's instruction by flow cytometer. Mitochondrial membrane potential was also evaluated b) using TMRM/PMPI after 48 hours of control/miR-+1 mimic transfection. The images were captured by confocal microscope and the quantification of fluorescent intensity was measured using FV1000 software (Olympus, Tokyo, Japan).

Determination of Cell Viability. Cell viability was measured by extracellular leakage of lactate dehydrogenase per manufacturer's instructions (Sigma Chemical St Louis, Mo., USA) as described. Cell viability was also determined by incubating cells with propidium iodide (PI) (2.5 mmol/L) in phosphate-buffered saline for 15 minutes at 37° C. and with 5% CO₂. Cell were washed twice with PBS after incubation and fluorescence intensity was determined by FACs in FL2 region using an Accuri C6 Flow Cytometer (Accuri, Ann Arbor, Mich.) at 530-nm excitation with a gated sample size of 10,000 cells.

HIF-1α Stabilization in Human Keratinocytes. Adenoviruses expressing a plasmid encoding a fusion protein of amino acids 1 to 529 of HIF-1α and the herpes simplex virus VP16 transactivation domain (pBABE-puro-HIF-1α-VP16) and a control plasmid encoding only VP16 (pBABE-puro-VP16) were used for transfecting the cells. HaCaT cells were grown in standard 12-well plates to 75% confluence. Next, cells were transfected with 2.3×10⁹ pfu Ad-VP16- HIF-1α or with the empty vector as control in 750 μL of media. Subsequently, 750 μL of additional media was added 4 h later and the cells were incubated for 72 h.

Western Blot Analysis. Immunoblotting was performed using HaCaT cell and wound tissue lysates. After protein extraction, the protein concentration was determined by BCA protein assay as described earlier. The samples (10-20 μg of protein/lane) were separated on a 4-12% SDS polyacrylamide gel electrophoresis and probed with rabbit polyclonal anti-notch1 (Dll1) antibody (1:1000 dilution, Abcam, Cambridge, Mass.), rabbit polyclonal anti-BAX antibody (1:1000 dilution, Abcam, Cambridge, Mass.), HRP conjugated anti-GAPDH (1:10,000 dilution). Bands were visualized by horseradish peroxidase-conjugated anti-rabbit-IgG raised in donkey and horseradish peroxidase-conjugated anti-mouse-IgG raised in sheep (Amersham Biosciences, Piscataway, N.J.) at 1:2,000 dilution and the enhanced chemiluminescence assay (Amersham Biosciences, Piscataway, N.J.).

Determination of ADP/ATP levels. Changes in the ADP/ATP ratio were measured using bioluminescent assay (EnzyLight™ ADP/ATP Ratio Assay Kit; BioAssay Systems) as described previously. The amount of ATP contained in the solution was determined using a bioluminescence assay kit. The luminescence produced in the reaction of ATP and luciferin was detected in a luminometer (Berthold Technologies U.S.A. LLC). The ATP content in each sample was corrected for the protein concentration that was determined with the bicinchoninic acid protein assay (Pierce).

Human subject: Human wound biopsy samples were obtained from chronic wound patients seen at OSU Comprehensive Wound Center (CWC) clinic. All human studies were approved by The Ohio State University's (OSU) Institutional Review Board (IRB). Declaration of Helsinki protocols was followed, and patients gave their written informed consent.

Animals and wound models. Male C57BL/6 mice were obtained from Harlan Laboratory. All animals were 8-10 weeks old at the time of experiment. All animal studies were performed in accordance with protocols approved by the Laboratory Animal Care and Use Committee of the Ohio State University and Indiana University. No statistical methods were used to predetermine sample size. Power analysis were not necessary for this study. The animals were tagged and grouped randomly using a computer-based algorithm (www.random.org). None of the mice with the appropriate genotype were excluded from this study. Mice were anesthetized by low-dose isoflurane inhalation as per standard recommendation. The dorsum was shaved, cleaned, and sterilized 48 h before the wounding. A bipedicle flap was developed. Flap edges were cauterized and then sutured to the adjacent skin. Full-thickness excisional wounds were developed in the middle of each flap with a 3-mm disposable biopsy punch. Two more wounds (control non-ischemic) were developed similarly in non-ischemic skin at the same cranio-caudal location. To study the kinetics of miR-1 expression, two non-ischemic 8×16 mm full-thickness excisional wounds were created on the dorsal skin, equidistant from the midline. Such wounds facilitate adequate separation of the different phases of wound healing to delineate the underline mechanisms with minimum contraction. Digital images of the wounds were taken on the days as indicated. Wound area measurement was done by digital planimetry using Image-J software (NIH), as described previously. The animals were euthanized at the indicated post-wounding time point and wound-edge tissues (1 mm away from the wound, snap frozen) or the wound tissues in optimal cutting temperature compound (OCT).

Tissue nanotransfection. Oligonucleotide delivery to the wound site was conducted via in vivo nanoelectroporation. Briefly, the LNA conjugate anti-miR-1 power inhibitor (Exiqon) (ACATACTTCTITACATTCCA; SEQ ID NO: 1) was diluted in PBS at a final concentration of 2.5 μM. A square wave electric pulse of 250 V in amplitude was then applied to the wound (AgilePulse, BTX) in order to electroporate the cell membranes and facilitate intracellular oligo delivery.

Laser Speckle Imaging. Perfusion imaging was performed using Laser Speckle Perfusion imaging system (Perimed Inc., Sweden). Color coded perfusion maps were acquired at all time points and average perfusion was calculated using PimSoft v1.4 software (Perimed Inc., Sweden). The wound edge and wound bed tissue regions were chosen as region of interests (ROI). From the real-time graphs obtained, time-of-interest (TOI) was chosen to include lower peak regions and to exclude motion related artifacts. Mean and standard deviation of perfusion data were obtained from the selected TOI perfusion data.

Immunohistochemistry. Immunostaining of K-14 (Covance; PRB-155P, 1:400) and Ki67 (Abcam; ab15580, 1:200) was performed on cryosections of wound sample using specific antibody as described previously. Briefly, OCT embedded tissue was cryosectioned at 10 μm thick, fixed with cold acetone, blocked with 10% normal goat serum, and incubated with specific antibodies against K14 (Covance; PRB-155P; 1:400) overnight at 4° C. Signal was visualized by subsequent incubation with either biotinylated-tagged or fluorescence-tagged appropriate secondary antibodies (Alexa 568-tagged α-mouse; Alexa 488-tagged α-rabbit, 1:200; Alexa 568-tagged α-rabbit, 1:200) for DAB and immunofluorescence staining.

Statistical analyses. In vitro data are reported as mean±SD of 3-6 experiments as indicated in respective figure legends. For animal studies, data are reported as mean±SD of at least 4-6 animals as indicated. Comparison between two groups was tested using Student's t test (two-tailed), whereas, the comparisons among multiple groups were tested using analysis of variance (ANOVA). p<0.05 was considered statistically significant.

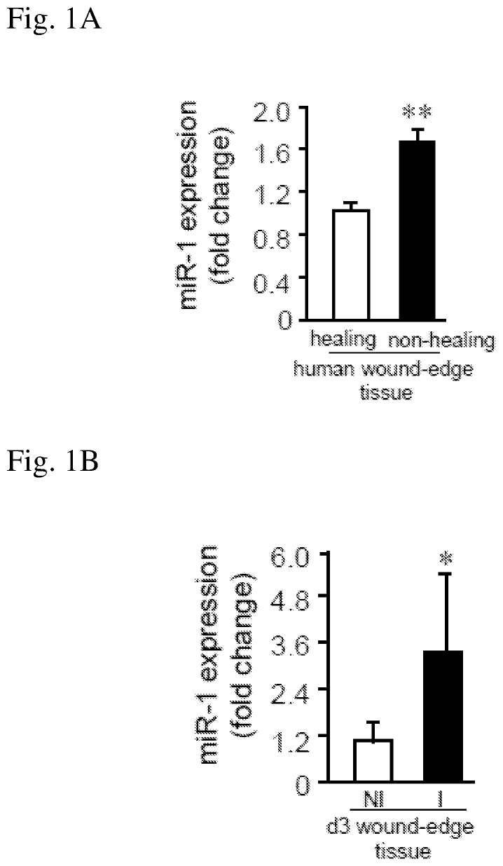

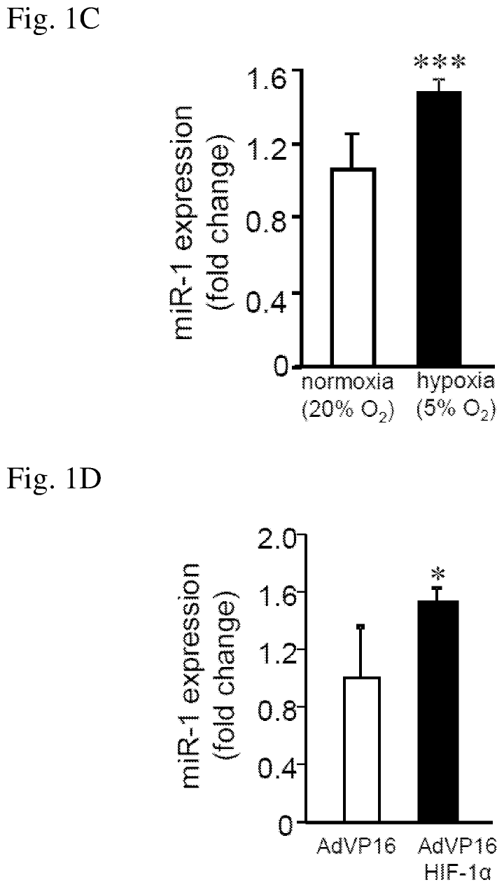

Ischemia induces miR-1 expression in keratinocytes. As shown in FIG. 1A, expression of miR-1 was significantly elevated at the wound-edge tissue of non-healing chronic wounds of human patients. To investigate mechanisms underlying ischemia-induced expression of miR-1, murine ischemic and non-ischemic wound-edge tissues were collected on day 3 following wounding of ischemic flap and non-ischemic skin in a bipedicle flap model. As shown in FIG. 1B, consistent with the observation in human chronic wound edge tissue, miR-1 expression was significantly elevated in murine ischemic wound-edge tissue compared to non-ischemic wound. Laser captured microdissection (LCM) of the murine skin also revealed significant induction of miR-1 in the epithelium.

Hypoxia is a subset of multiple factors involved in tissue ischemia. To test the isolated contribution of hypoxia in miR-1 induction, human keratinocytes were cultured under normoxic and hypoxic conditions. FIG. 1C illustrates keratinocyte miR-1 is induced in response to hypoxia, thus establishing that miR-1 is properly characterized as a hypoxamiR. In mammalian cells, response to hypoxia may be broadly classified into those that are dependent or independent on Hypoxia-inducible factor 1-alpha (HIF-1a). To test whether hypoxia-induced expression of keratinocyte miR-1 was dependent on HIF-1α, cells were subjected to Ad-VP16-HIF-1α gene delivery causing stabilization of the transcription factor even under conditions of normoxia. With reference to FIG. 1D, stabilization of HIF-1α protein under conditions of normoxia was sufficient to cause elevated expression of miR-1. Thus, in keratinocytes, induction of miR-1 under conditions of hypoxia is HIF-1α dependent.

HIF inducible miR-1 targets Delta-like protein 1 (Dll1).

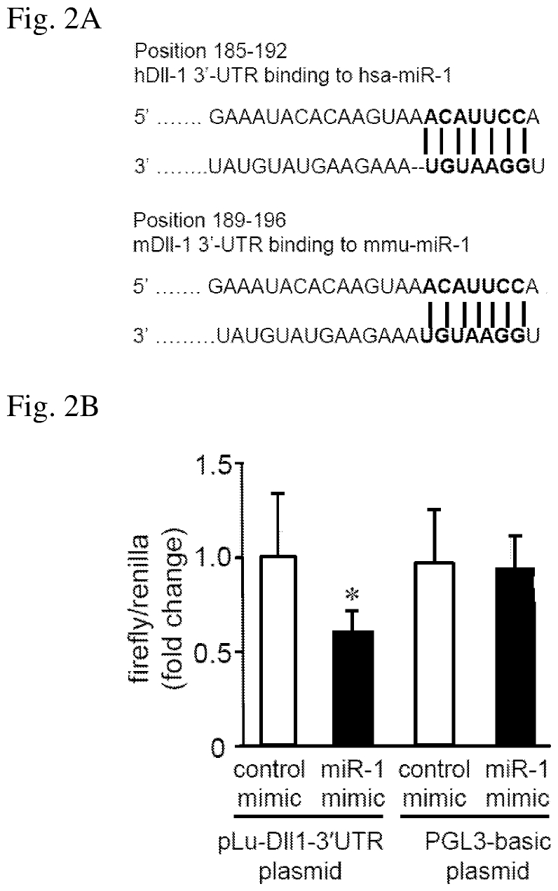

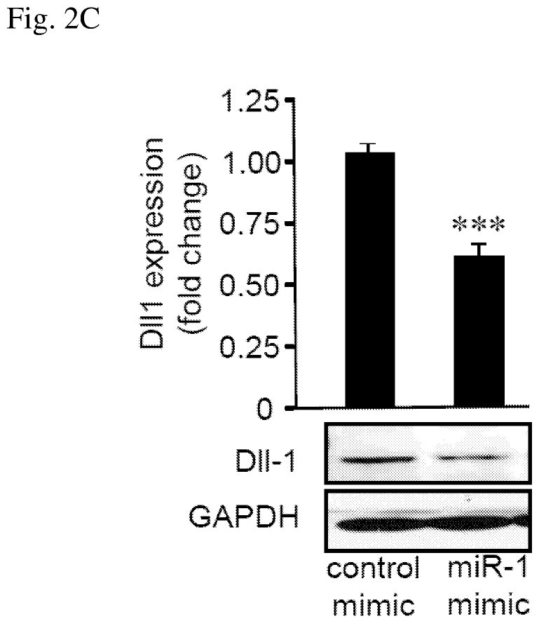

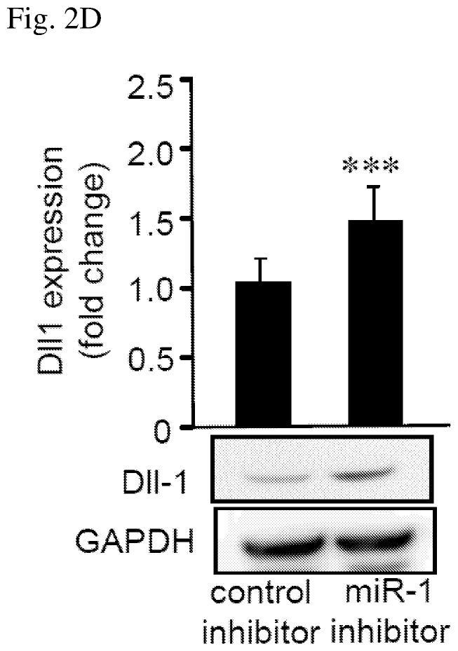

With reference to FIG. 2A, in silico analysis by using TargetScan, miRanda, and Diana-MicroT algorithms predicted that 3′-UTR of Dll1 is subject to post-transcriptional gene silencing by miR-1. With reference to FIG. 2B, delivery of miR-1 mimic oligonucleotide to human keratinocytes blunted Dill 3′-UTR luciferase reporter activity and subsequently lowered Dll1 protein abundance (see FIG. 2C). Suppression of miR-1 using a miR-1 inhibitor oligonucleotide, significantly increased Dll1 protein abundance (see FIG. 2D) establishing that in human keratinocytes Dll1 is a direct target of miR-1.

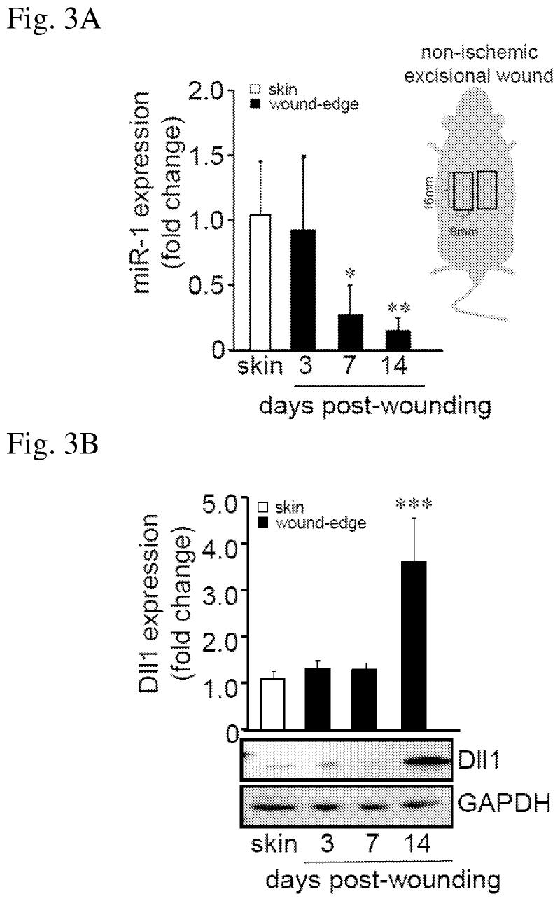

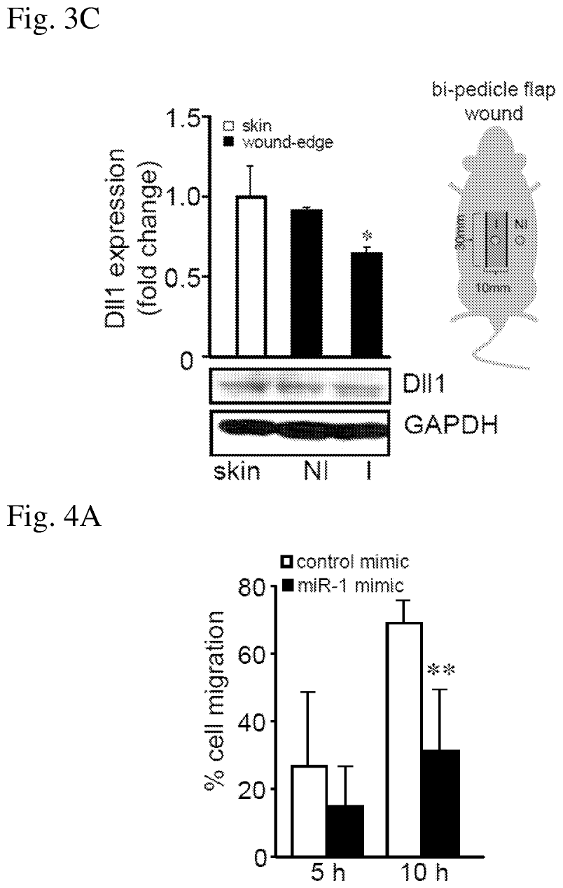

Normal wound healing requires lowering of miR-1 and upregulation of Dll1 in the wound-edge tissue. To study the significance of miR-1 in wound healing, miR-1 expression was studied at the wound-edge tissue at different time points post-wounding. In an established murine model of non-ischemic excisional wound, induction of injury progressively lowered wound-edge miR-1 and increased Dll1 in a time dependent manner (see FIGS. 3A and 3B). However, under conditions of ischemia such as in a bi-pedicle ischemic flap model, such responses were impaired. Ischemic injury, in contrast to what was evident under conditions of non-ischemic wound healing, increased wound-edge miR-1 and lowered Dll1 (see FIG. 3C).

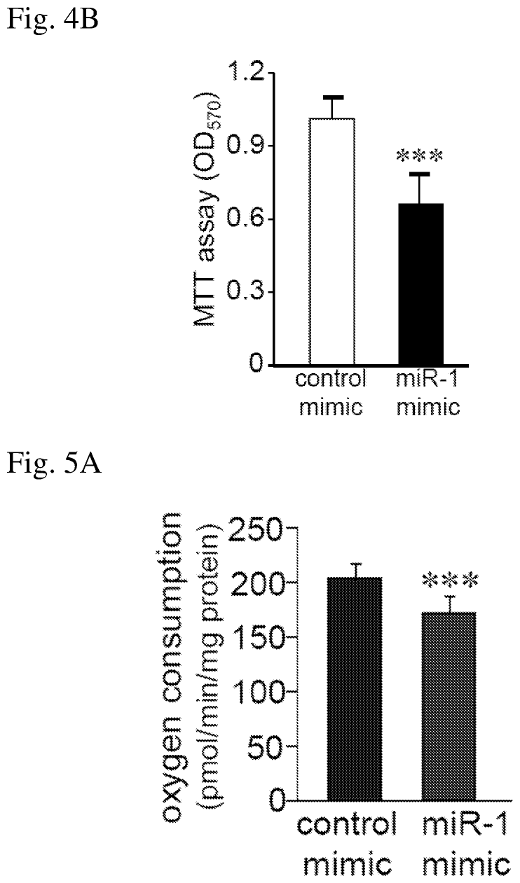

Elevated miR-1 impaired keratinocyte proliferation and migration Keratinocyte migration is impaired under conditions of healing complicated by ischemia. To elucidate the role of miR-1 in keratinocyte migration, we performed a scratch assay on human keratinocytes. Delivery of miR-1 mimic resulted in significant impairment of keratinocyte migration (FIG. 4A). Furthermore, delivery of miR-1 mimic limited keratinocyte proliferation (FIG. 4B). Taken together, these data support the contention that induction of miR-1 by factors such as the hypoxia component of ischemia is likely to be in conflict with wound closure.

High miR-1 Limits Mitochondrial Function

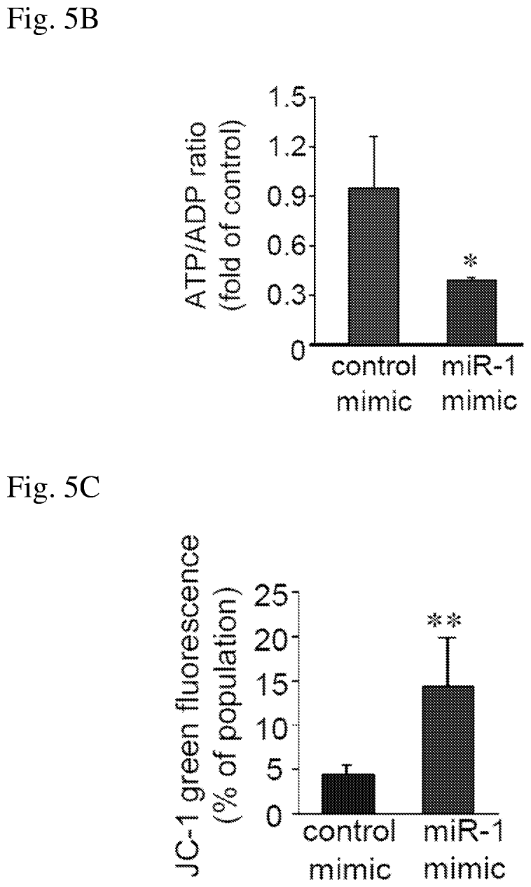

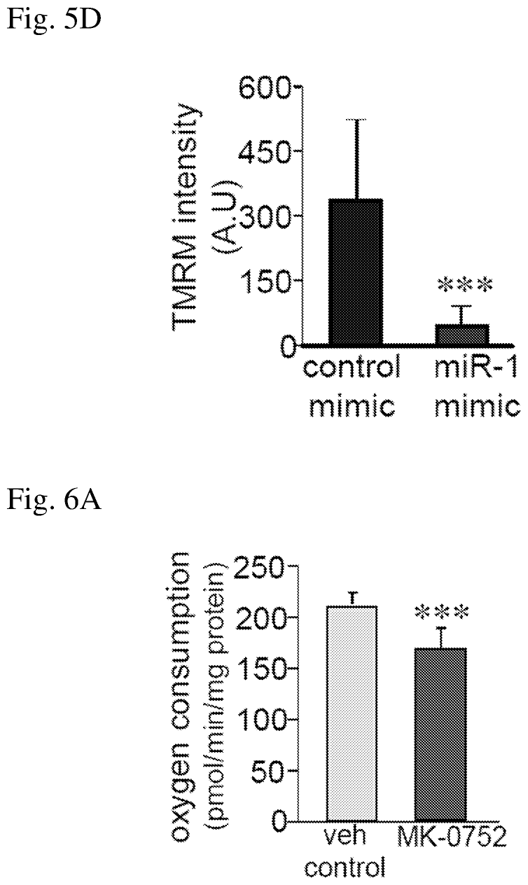

Direct assessment of keratinocyte mitochondrial respiration showed decreased rate of oxygen consumption in response to elevated miR-1 (FIG. 5A). High miR-1 caused increased dimerization of BAX, a known trigger of mitochondrial depolarization. That miR-1 blunted oxidative metabolism was evident from the observation that in keratinocytes elevated miR-1 sharply lowered ATP/ADP (FIG. 5B). Mitochondrial depolarization following elevated miR-1 was manifested as decreased mitochondrial membrane potential (ΔΨ)). Compromised ΔΨ, under conditions of elevated miR-1, was further assessed by TMRM and PMPI staining. Significant decrease in TMRM fluorescence was observed in keratinocytes subjected to the delivery of miR-1 mimic (FIG. 5C).

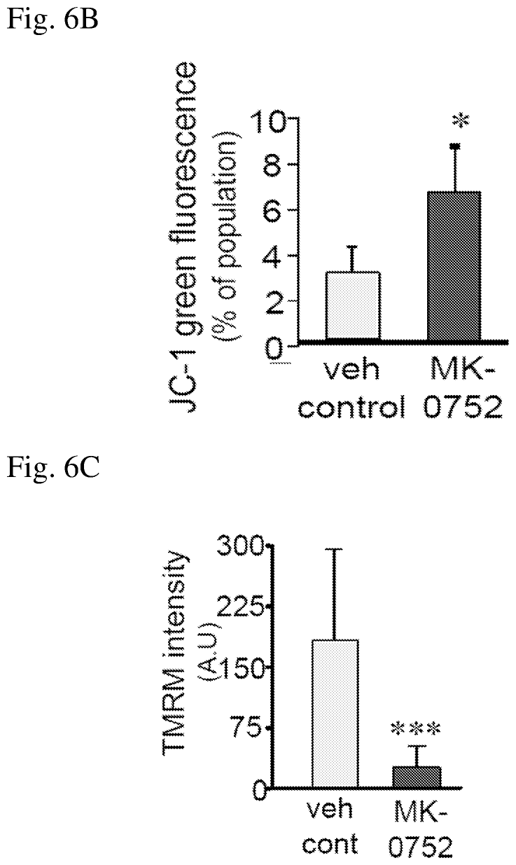

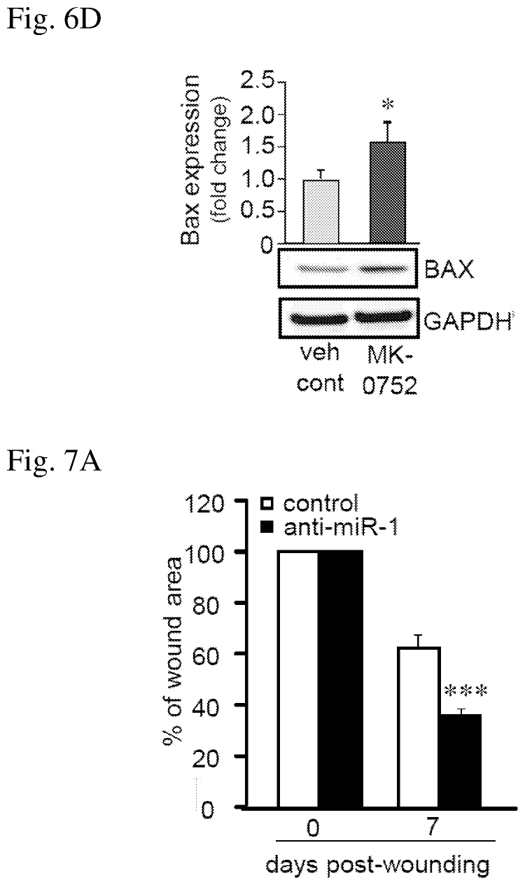

Pharmacological inhibition of Dll1 induced mitochondrial dysfunction. To test the significance of the Dll1-Notch signaling pathway in keratinocytes, a synthetic small molecule inhibitor MK0752 was tested. Such inhibition of the Notch signaling pathway decreased rate of oxygen consumption, a measure of mitochondrial respiration (FIG. 6A). MK0752 treatment also lowered ΔΨ causing mitochondrial depolarization as evident from studies using TMRM and PMPI. Pharmacological inhibition of Notch signaling caused BAX dimerization (FIG. 6C). Taken together, it was clear that the Notch signaling pathway is essential for mitochondrial function.

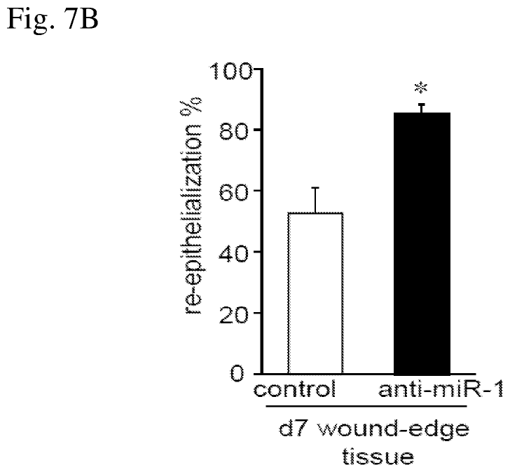

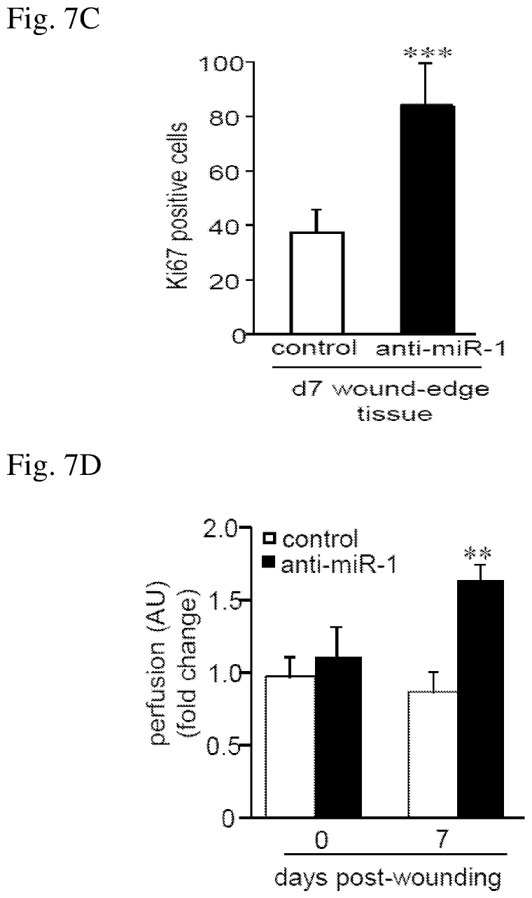

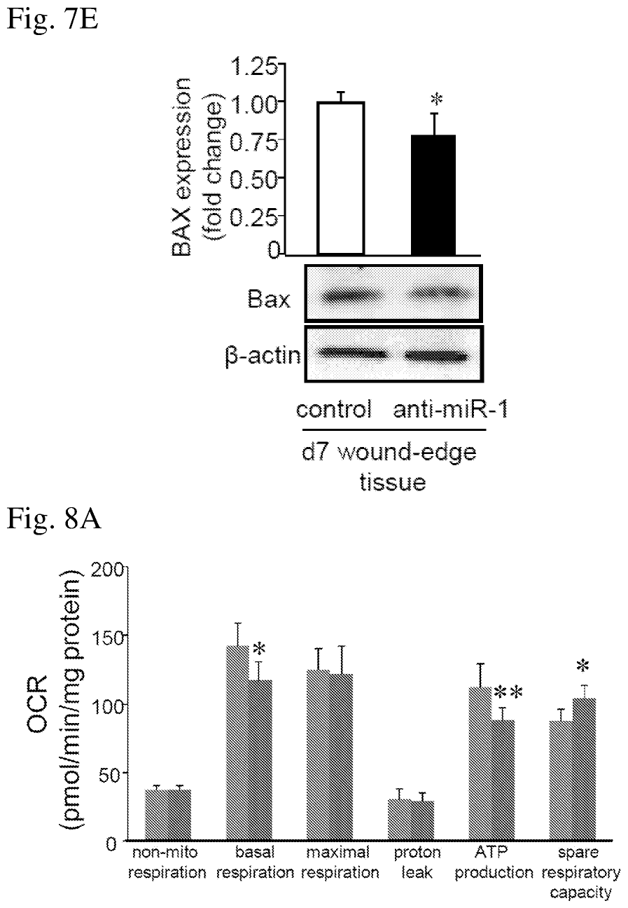

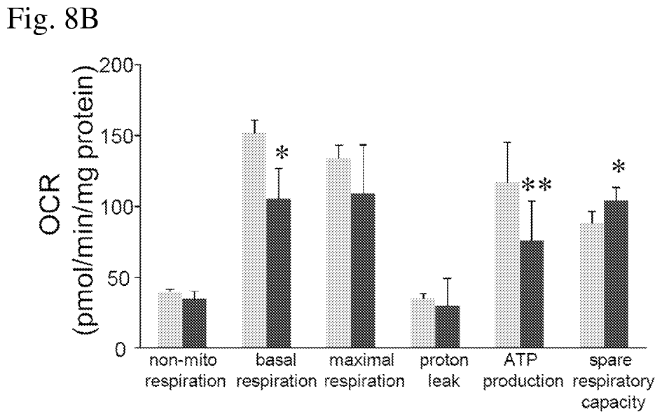

LNA conjugated anti-miR-1 rescued ischemic wound closure. Standardized ischemic wounds were developed on the dorsal skin of C57BL/6 mice as described previously. Sequestration of excessive miR-1 from the wound-edge tissue was achieved using tissue nanotransfection. Such lowering of miR-1 significantly accelerated ischemic wound closure (FIG. 7A). Consistently, sequestration of wound-edge miR-1 accelerated re-epithelialization of ischemic wounds (Fig. B). Associated with such improved healing responses were increased cell proliferation in the basal region of the wound epithelium (FIG. 7C). That miR-1 sequestration improved the quality of healing was also manifested as improved cutaneous perfusion (Fig D). Improved mitochondrial health was indicated by lower BAX dimerization in the wound-edge of miR-1 sequestered tissue (FIG. 7E). Taken together, sequestration of excessive miR-1 induced in the ischemic wound-edge tissue emerged as a viable strategy to rescue healing of ischemic cutaneous wounds.

Various modifications and additions can be made to the embodiments disclosed herein without departing from the scope of the disclosure. For example, while the embodiments described above refer to particular features, the scope of this disclosure also includes embodiments having different combinations of features and embodiments that do not include all of the described features. Thus, the scope of the present disclosure is intended to embrace all such alternatives, modifications, and variations as fall within the scope of the claims, together with all equivalents.

All publications, patents and patent applications referenced herein are hereby incorporated by reference in their entirety for all purposes as if each such publication, patent or patent application had been individually indicated to be incorporated by reference.

1. A method of accelerating wound closure in a subject, said method comprising the step of decreasing the concentration of functional miR-1 in the cells of wound-edge tissue.

2. The method of claim 1 wherein the wound is an ischemic cutaneous wound.

3. The method of claim 2 wherein the wound to be treated is a chronic wound in a diabetic patient.

4. The method of claim 1 wherein an inhibitor of miR-1 is administered to wound-edge tissue in an amount effective to lower miR-1 activity and increase Dll1 activity.

5. The method of claim 4 wherein the miR-1 inhibitor is an oligonucleotide at least 8 nucleotides in length, wherein the oligonucleotide has at least 85% complimentary sequence identity to a continuous 8 nucleotide sequence of human mature miR-1 sequence (UGGAAUGUAAAGAAGUAUGUAU; SEQ ID NO: 1) or a complement thereof.

6. The method of claim 5 wherein said oligonucleotide is an RNA comprising a locked nucleic acid.

7. The method of claim 6 wherein said locked nucleic acid is the N-terminal or C-terminal nucleotide in said oligonucleotide.

8. The method of claim 6 wherein said oligonucleotide comprises a locked nucleic acid at the N-terminus and the C-terminus of said oligonucleotide.

9. The method of claim 5 wherein functional miR-1 concentrations are decreased by transfecting cells with said oligonucleotide.

10. The method of claim 9 wherein an anti-miR-1 oligonucleotide is delivered into the cytosol of human epidermal and dermal cells.

11. The method of claim 10 wherein the oligonucleotide is delivered into the cytosol of cells via skin electroporation or tissue nanotransfection.

12. A pharmaceutical composition for enhancing wound closure, said composition comprising
an oligonucleotide at least 8 nucleotides in length, wherein the oligonucleotide has at least 85% complimentary sequence identity to a continuous 8 nucleotide sequence of human mature miR-1 sequence (SEQ ID NO: 1) or a complement thereof; and
a pharmaceutically acceptable carrier.

13. The composition of claim 12 wherein said oligonucleotide is an RNA comprising a locked nucleic acid.

14. The composition of claim 13 wherein said locked nucleic acid is the N-terminal or C-terminal nucleotide in said oligonucleotide.

15. The composition of claim 13 wherein said oligonucleotide comprises a locked nucleic acid at the N-terminus and the C-terminus of said oligonucleotide.

16. A method to promote wound healing in a subject, the method comprising the step of administering a miR-1 inhibitor to a wound on said subject, wherein the miR-1 inhibitor is an oligonucleotide at least 8 nucleotides in length, wherein the oligonucleotide has at least 95% sequence identity to a continuous 8 nucleotide sequence of human mature miR-1 sequence (UGGAAUGUAAAGAAGUAUGUAU; SEQ ID NO: 1) or a complement thereof.

17. The method of claim 16 wherein the administration of the miR-1 inhibitor to the wound reduces function or activity of miR-1, thereby promoting wound healing.

18. The method of claim 1, where the miR-1 inhibitor is an oligonucleotide having has at least 95% sequence identity to a continuous nucleotide sequence, at least 8 nucleotides in length, of human mature miR-1 sequence (SEQ ID NO: 1) or a complement thereof.

19. The method of claim 16, where the miR-1 inhibitor is an oligonucleotide comprising an amino acid sequence identical to a continuous 8 nucleotide sequence of human mature miR-1 sequence (SEQ ID NO: 1) or a complement thereof.

20. The method of claim 16 wherein the miR-1 inhibitor is an RNA comprising the sequence ACAUUCCA (SEQ ID NO: 2), or its complement, or the corresponding DNA ACATTCCA (SEQ ID NO: 3), or its complement).
